(19) United States
(12) Patent Application Publication
(10) Pub. No.: US 2008/0300473 A1
(43) Pub. Date: Dec. 4, 2008

(54) Stabilized Multi-Wavelength Laser System for Non-Invasive Spectrophotometric Monitoring

(75) Inventor: Paul Benni, Guilford, CT (US)

Correspondence Address: O''Shea Getz P.C., 1500 MAIN ST. SUITE 912, SPRINGFIELD, MA, 01115, US

(73) Assignee: CAS Medical Systems , Inc., Branford, CT (US)

(21) Appl. No.: 12/097,438

(22) PCT Filed: Dec. 14, 2006
(86) PCT No.: PCT/US2006/062108

§ 371 Date: Jun. 25, 2008

Related U.S. Application Data
(60) Provisional application No. 60/751,008, filed on Dec. 16, 2005.
(60) Provisional application No. 60/844,435, filed on Sep. 14, 2006.

(51) Int. Cl. A61B 5/1455

(52) U.S. Cl. 600/323

(57) ABSTRACT

A spectroscopy system that may be used for spectrophotometric oxygenation monitoring of tissue includes a monitor portion and a sensor portion. The sensor portion generally includes a light source and one or more light detectors. The sensor portion may attach to a human to sense light signals from the light source that have traversed biological tissue, the light signals ultimately being used by the system to determine biological tissue blood hemoglobin oxygenation levels. The monitor portion generally includes a processor and a visual display. A laser beam combiner may couple a plurality of laser diode output light signals into one optical fiber. To stabilize the output of each of the laser diodes, an optical fiber light stabilizer is coupled to the combined laser diode output. The optical fiber light stabilizer redistributes the modes in the optical fiber such that the higher-order modes are filled until an equilibrium mode distribution is established. A light sensor may also provide feedback with respect to the laser diode output, which allows for compensation of any laser diode light output instability independently of optical fiber related instabilities.

Applicant hereby claims priority benefits under 35 U.S.C. § 119(e) of U.S. Provisional Patent Application No. 60/751,008 filed Dec. 16, 2005 and U.S. Provisional Patent Application No. 60/844,435 filed Sep. 14, 2006, the disclosures of which are herein incorporated by reference.

This invention was made with Government support under Contract No. 2R44NS45488-01 awarded by the Department of Health & Human Services. The Government has certain rights in the invention.

### BACKGROUND OF THE INVENTION

1. Technical Field

This invention relates in general to apparatus and methods for non-invasively examining biological tissue utilizing near-infrared spectroscopy techniques, and in particular to a relatively stabilized laser diode light source for use with such apparatus and methods.

2. Background Information

Near-infrared spectroscopy (NIRS) is an optical spectrophotometric method that can be used to continuously monitor biological tissue characteristics such as the oxygenation level within the tissue. The NIRS method is based on the principle that light in the red/near-infrared range (660-1000 nm) can pass easily through skin, bone and other tissues where it encounters hemoglobin located mainly within micro-circulation passages; e.g., capillaries, arterioles, and venuoles. Hemoglobin exposed to light in the near-infrared range has specific absorption spectra that vary depending on its oxygenation state; i.e., oxyhemoglobin (HbO₂) and deoxyhemoglobin (Hb) each act as a distinct chromophore. By using light sources that transmit near-infrared light at specific different wavelengths, and by measuring changes in transmitted or reflected light attenuation, concentration changes of the oxyhemoglobin and deoxyhemoglobin can be monitored as well as total or absolute values of tissue oxygenation levels can be determined or calculated. The ability to continually monitor or determine cerebral oxygenation levels, for example, is particularly valuable for those patients subject to a condition in which oxygenation levels in the brain may be compromised, leading to brain damage or death.

An NIRS system typically includes a sensor portion having a light source and one or more light detectors for detecting reflected and/or transmitted light. The light signal is created and sensed in cooperation with the overall NIRS system that includes a monitor portion having a computer or processor that runs an algorithm for processing signals and the data contained therein to, for example, calculate or determine the hemoglobin oxygenation concentration or saturation levels. Typically the monitor portion is separate from the sensor portion. Light sources such as light emitting diodes (LEDs) or laser diodes that produce light emissions in the wavelength range of 660-1000 nm are typically used. Each light source produces an infrared light signal at a particular wavelength at which a known absorption response is produced depending on the amount of oxygen concentration in the hemoglobin. Several different specific wavelengths are typically employed, for example, at 690 nm, 780 nm, 805 nm, and 850 nm. Thus, a corresponding number of light sources are employed in the sensor portion, with these light sources usually being located together. One or more photodiodes or other types of light detectors detect light reflected from or passed through the tissue being examined, and oftentimes the photodiodes are located at specific, predetermined different distances from the light source location. The NIRS system processor cooperates with the light source and detector to create, detect and analyze the signals, for example, in terms of their intensity and wave properties. U.S. Pat. Nos. 6,456,862 and 7,072,701, both of which are hereby incorporated by reference in their entirety, each disclose an NIRS system (e.g., a cerebral oximeter) and a methodology for analyzing the signals within the NIRS system to produce an indication of tissue oxygenation levels to a system user, typically a clinician.

However, a spectrophotometric system such as a cerebral oximeter that utilizes a laser system containing one or more laser diodes may demonstrate instability in operation for various reasons. For example, the output of the laser system may become unstable over time in terms of its wavelength and power output due to various factors, including environmental (e.g., temperature). Also, oftentimes the individual laser diode(s) is located apart from the sensor portion of the overall system and, as such, the laser diode output may be coupled directly by an optical fiber to the sensor portion. Therefore, problems may exist, for example, in the connection or coupling of the laser diode output to the optical fiber, for example, due to stripped cladding of the optical fiber, improper centering of the optical fiber in the connector, or use of an improper connector. Also, instabilities in the optical fiber itself may exist, for example, due to bending, temperature, and mode variance. In general, it is known that when the sensor portion of the spectrophotometric system is directly connected to the laser diode light source by an optical fiber of a few meters in length, an unstable light output can occur. Any sufficient degree of instability in the overall laser system output can cause corresponding errors in the overall spectrophotometric system, particularly those that utilize differential wavelength algorithms.

What is needed, therefore, is a laser system light source that contains multiple laser diodes, light emitting diodes (LEDs), or other similar electro-optical light sources that can provide different discrete wavelengths for use in a spectrophotometric system such as a cerebral oximeter, where the laser system provides a relatively stable and consistent light radiation output in terms of output parameters such as, for example, power, intensity and radiation pattern.

### SUMMARY OF THE INVENTION

A spectroscopy system that may be used for spectrophotometric monitoring of tissue includes a monitor portion and a sensor portion. The spectroscopy system is described hereinafter as a cerebral oximeter operable to monitor brain tissue. The spectroscopy system is not limited to a cerebral oximeter embodiment, however, and may be utilized in other spectroscopic applications. The sensor portion generally includes a light source and one or more light detectors. The sensor portion may attach to a human to sense light signals from the light source that have traversed biological tissue, the light signals ultimately being used by the system to determine biological tissue blood hemoglobin oxygenation levels. The monitor portion generally includes a processor for determining or calculating tissue oxygenation levels from the sensed light signals, together with a visual display to indicate the determined oxygenation levels in various forms. The light source may comprise a plurality of laser diodes, LEDs, or the like, each providing infrared light at a particular wavelength. A laser beam combiner may couple the plurality of laser diode output light signals into one optical fiber. To stabilize the output of each of the laser diodes in the laser beam combiner, an optical fiber light stabilizer is coupled to the combined laser diode output. The light stabilizer may include several meters of multimode optical fiber wrapped around a circular spool. The optical fiber coupled to a laser diode is typically “underfilled” when the laser light enters the optical fiber (i.e., usually only the lower-order modes or paths are utilized in the optical fiber) since the laser diode radiation output has a lower numeral aperture (NA) compared to the optical fiber. The optical fiber light stabilizer redistributes the modes so that the higher-order modes are filled until an equilibrium mode distribution is established. The modes nearest to the axis of the fiber core are referred to as the lower-order modes, while the paths with the relatively greatest deviation (i.e., highest angles from the core axis) are referred to as the higher-order modes. The resultant laser system light output typically demonstrates a relatively high degree of stability when modal equilibrium is achieved. A light sensor (e.g., a photodiode) may also provide feedback with respect to the laser diode output, which allows for compensation of any laser diode light output instability independently of optical fiber related instabilities.

With an underfilled optical fiber, the light may “jump” between lower and higher modes due to temporary fiber bending or temperature changes, which causes instability in the laser system output. The optical fiber light stabilizer corrects this problem by redistributing the light into an equilibrium mode distribution. Such an equilibrium mode distribution may also be achieved with a relatively large amount (e.g., 1000-2000 meters) of uncoiled optical fiber. For example, a laser diode connected to a multimode optical fiber cable a few meters in length with a numeral aperture (NA) of 0.22 (conic light output of 12.7 degrees) will fill only the lower modes, resulting in an output NA of 0.18 (10.4 degrees) or less, depending on the laser light launch NA. By attaching the laser diode to the optical fiber light stabilizer comprising the same optical fiber but at a longer length (e.g., 20 meters) wrapped around a spool with a radius that is at least approximately equal to (e.g., or slightly larger than) the minimum long term bend radius of the optical fiber, the light output will reach an equilibrium mode distribution with an output NA of approximately 0.22 (12.7 degrees), resulting in a relatively more stable output.

These and other features and advantages of the present invention will become apparent in light of the drawings and detailed description of the present invention provided below.

### BRIEF DESCRIPTION OF THE DRAWINGS

FIG. 1 is a simplified diagrammatic representation of an NIRS system sensor portion placed on the head of a human subject and coupled to an NIRS system monitor portion.

FIG. 2 is a diagrammatic cross-section of the NIRS system sensor portion of FIG. 1.

FIG. 3 is a diagrammatic representation of an embodiment of a portion of the NIRS system of FIG. 1 including a combined multiple laser diode light source and associated components.

FIG. 4 is a detailed diagrammatic representation of an optical fiber light stabilizer assembly within the NIRS system of FIG. 3.

FIG. 5 is a diagrammatic representation of an embodiment of a portion of the NIRS system of FIG. 1 including a combined multiple laser diode light source and associated components.

### DETAILED DESCRIPTION OF THE INVENTION

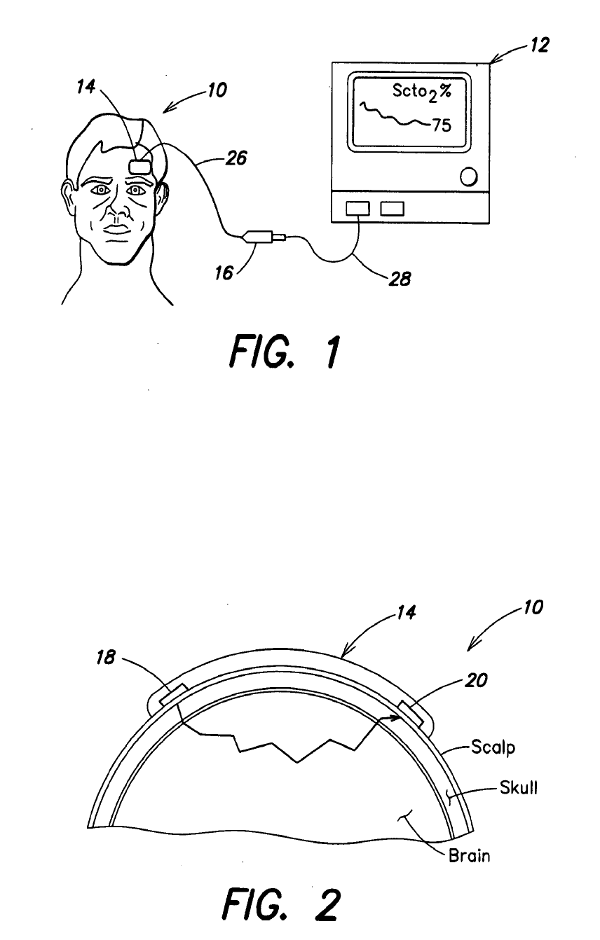

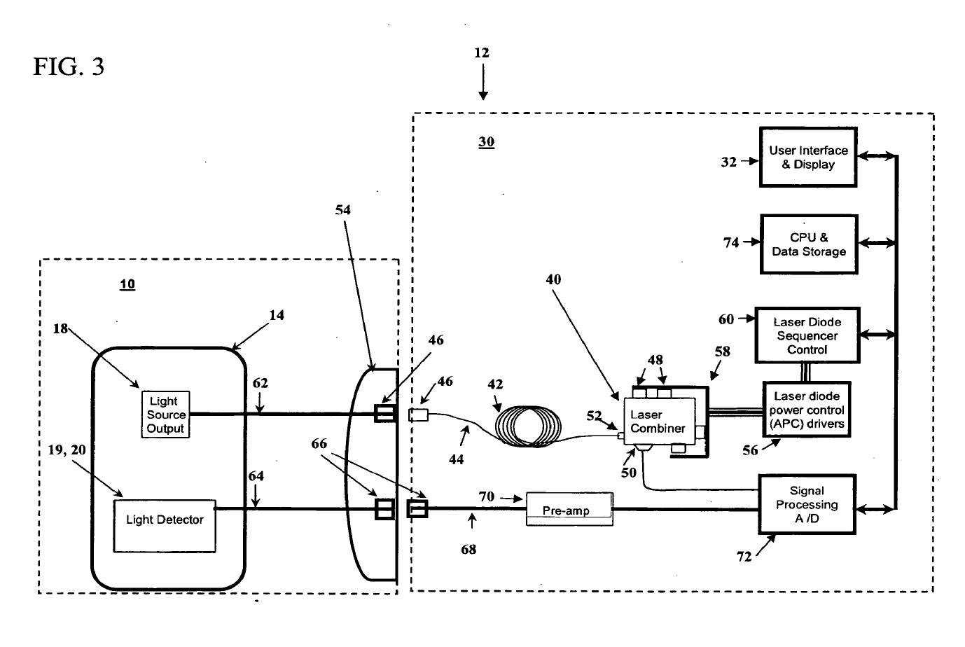

Referring to FIGS. 1 and 2, an NIRS spectrophotometric system for use with the present invention may be similar to that described and illustrated in the aforementioned U.S. Pat. Nos. 6,456,862 and 7,072,701. However, it should be understood that the present invention is not limited to use with the spectrophotometric systems of these patents, or with any specific spectrophotometric system. Instead, the present invention may be utilized with various types of spectroscopy apparatus or methods that include one or more laser diodes, LEDs, or other light emitting electro-optical components as light sources. The spectrophotometric system of FIGS. 1 and 2 generally includes a sensor portion 10 and a monitor portion 12. The sensor portion 10 may include one or more sensor assemblies 14 and a connector housing 16. Each sensor assembly 14, which may be a flexible structure that can be attached directly to a location (e.g., the head) on a human subject, may include a light source 18 and one or more light detectors 20. FIG. 2 diagrammatically illustrates a sensor assembly 14 having a single detector 20. An example of an acceptable sensor assembly having more than one detector can be found in PCT Application No. PCT/US06/41268 filed on Oct. 18, 2006, which application is commonly assigned with the present application, and which is hereby incorporated by reference in its entirety. A disposable adhesive envelope or pad may be used for mounting the sensor assembly 14 easily and securely to the skin of the human subject under test. The light source 18 may comprise a plurality of laser diodes that in general emit a light signal at a narrow spectral bandwidth at known but different wavelengths (e.g., 690 nm, 780 nm, 805 nm, and 850 nm). The laser diodes may be mounted within the sensor assembly 14 to provide the output of the light source 18. Alternatively, the laser diodes may be located within the monitor portion 12, as described in more detail hereinafter with respect to FIG. 3. If located within the monitor portion 12, the laser diodes have their light output transported to the sensor assembly 14 within the sensor portion 10 by way of an optical fiber cable. A first connector cable 26 connects each one of the sensor assemblies 14 to the connector housing 16, and a second connector cable 28 connects the connector housing 16 to the monitor portion 12. The light detector 20 may comprise photodiodes. Depending on the location of the laser diodes (i.e., in the sensor portion 10 or in the monitor portion 12), the connector cables 26, 28 may comprise only electrical cables or a combination of electrical and optical fiber cables. The monitor portion 12 may include an internal computer processor for processing light intensity signals from the light detector 20 in accordance with various algorithms, for example those described in the aforementioned U.S. Pat. Nos. 6,456,862 and 7,072,701. The spectrophotometric system monitor portion 12 may include a display screen for visually displaying various types of information (e.g., the determined oxygen concentration or saturation levels) to the system user (e.g., a clinician).

Referring to FIG. 3, the spectrophotometric system monitor portion 12 includes various components, among them being a multiple laser beam combiner 40, an optical fiber light stabilizer 42, a predetermined length of multimode optical fiber 44, and an optical fiber connector coupler 46 (e.g., an ST-type connector coupler). The multiple laser beam combiner 40 includes a plurality of laser diodes 48, a laser output monitor photodiode 50, and a fiber optic connector 52 (e.g., an SMA-type connector). An example of an acceptable combiner 40 is the multiple laser beam combiner provided by Princetel, Inc. of Lawrenceville, N.J., U.S.A. The Princetel laser beam combiner 40 typically has three or four laser diodes 48, and all of the laser diode light output signals are combined into a single laser beam or output light signal using beamsplitters and polarizing filters within the combiner 40. A lens inside the combiner 40 focuses the laser light output into the optical fiber 44 via the SMA connector 52. Alternative versions of the fiber optic connector 52 could be used, such as an APC connector, which reduces back reflection of light entering back into the laser combiner 40, potentially causing interference to laser diode power control and monitoring. An APC connector has an angled (e.g. about 8 degrees) polished fiber optic face, which redirects back reflected light in a different direction or axis from the output light signal that is entering into the APC connector by internal reflection. For example, an SMA connector could be polished at an angle of 8 degrees to function as an APC connector. The NIRS sensor assembly 14 optically interfaces to the spectrophotometric system monitor portion 12 via the optical fiber connector coupler 46, which may be part of a detachable connector 54 that connects the monitor portion 12 with the sensor portion 10. The detachable connector 54 may be part of the connector housing 16 of FIG. 1, or may be separate therefrom. During operation, the laser diodes 48 may be pulsed one at a time (time multiplexed) or pulsed at different frequencies (frequency multiplexed) and their light outputs are optically coupled to the multimode optical fiber 44 via the fiber optic connector 52. Laser light optically coupled to the multimode optical fiber 44 characteristically has a lower NA compared to that of the multimode optical fiber 44, and therefore the laser light usually underfills the modal structure of the optical fiber 44. The laser light propagates through the optical fiber light stabilizer 42, which effectively establishes equilibrium modal distribution in the multimode optical fiber 44. Equilibrium modal distribution is typically defined as the condition in a multimode optical fiber 44 where after light propagation has taken place for a certain distance down the fiber 44, known as the “equilibrium length,” the relative power distribution among modes becomes statistically constant and remains so for the duration of further propagation down the optical fiber. After the equilibrium length has been traversed, the NA of the output of the optical fiber 44 is independent of the NA of the optical source (e.g., the laser diode) that sends light down the optical fiber. The laser light then propagates to the NIRS sensor assembly 14 through the remaining portion of the multimode optical fiber 44 and through the optical fiber connector coupler 46 to the sensor portion 10.

The laser diodes 48 are electrically actuated by laser diode power control drivers 56 via an electrical cable harness 58. A laser diode sequencer control 60 connects to the laser diode drivers 56 to provide laser diode pulse timing and control. The laser light from the multiple laser beam combiner 40 propagates through the optical fiber light stabilizer 44 and through the optical fiber connector coupler 46 to the NIRS sensor assembly 14. In the sensor assembly 14, the laser diode light propagates through a single core multimode optical fiber cable 62. The laser diode light is emitted out of the sensor assembly 14 at the light source output 18 and into the human subject (FIG. 1). The light detector 20 of the NIRS sensor assembly 14 receives the light after it has passed through the human subject being monitored via transmission and/or reflectance. The light detector 20 is electrically connected to a shielded cable 64 which interfaces with the NIRS monitor portion 12 via a shielded cable coupler 66. In some embodiments, the electrical signals received from the light detector 20 on a line 68 are electrically processed and amplified by a pre-amplifier 70 and by a signal processor 72, which may include an analog-to-digital converter. The signal processor 72 and CPU or monitor processor 74 convert the received signals into physiological parameters by various spectrophotometric methods (e.g., those of U.S. Pat. Nos. 6,456,862 and 7,072,701), and the resultant physiological parameters (e.g., tissue oxygenation concentration or saturation levels) may be visually displayed on the user display 32. Also, light sampled by the laser output monitor photodiode 50 could be used as the input intensity (Io) signal utilized in spectrophotometric type algorithms such that described in U.S. Pat. No. 6,456,862.

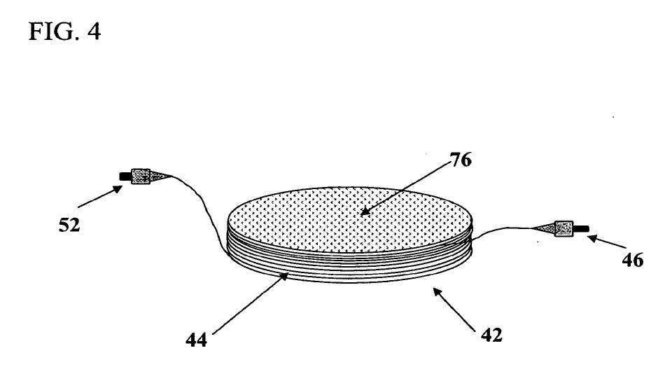

Referring to FIG. 4, there illustrated in more detail is the optical fiber light stabilizer 44, which may comprise the multimode optical fiber 44 wrapped around a circular spool 76. This may be carried out in a manner similar to the mandrel wrapping technique, which is typically used in multimode fiber optics to modify the modal distribution of a propagating optical signal. A cylindrical rod wrap includes a specified number of turns of optical fiber on a mandrel or spool of a predetermined size, depending on the fiber characteristics and the desired modal distribution. Mandrel wrapping has application in optical transmission performance tests, to simulate or establish equilibrium mode distribution in a launch fiber (i.e., an optical fiber used to inject a test signal in another optical fiber under test). If the launch optical fiber is fully filled ahead of the mandrel wrap, the higher-order modes will be stripped off, leaving only the lower-order modes. If the launch optical fiber is underfilled, for example, as a consequence of being energized by a laser diode, there will be a redistribution to higher-order modes until modal equilibrium is reached. The spool 76 may have a radius that is at least approximately equal to the long term bend radius of the multimode optical fiber 44. One end of the multimode optical fiber 44 may be terminated by the fiber optic connector 52, and the other end of the multimode optical fiber 44 may be terminated by the optical fiber connector coupler 46. Other alterations besides that described above in a multimode optical fiber could be carried out to achieve equilibrium mode distribution. For example, a short segment of optical fiber placed in a rigid apparatus that applies pressure on the fiber in different locations to cause microbends may be used, where such microbends induce redistribution of the modes to fill the higher-order modes until an equilibrium mode distribution is established. Also, a combination of lenses may be used to achieve similar results.

The optical fiber light stabilizer 42, which is relatively rugged mechanically, provides for a relatively stable and consistent laser diode light output in terms of parameters such as power, intensity, and radiation pattern, which helps to ensure accuracy of NIRS system monitored parameters. For example, the relatively high degree of output light stability allows for accurate differential wavelength tissue oxygenation signal processing, such as that described in the aforementioned U.S. Pat. No. 6,456,862. Due to the increased output light stability, another advantage is that the discrete laser diode light output wavelengths may be spaced relatively closer together, which provides for relatively accurate tissue oxygenation spectrophotometric measurement, despite the closer wavelength dependent light absorption coefficient values. Closer spaced wavelengths also allow for relative reduction of wavelength dependent light pathlength differences, which may cause errors in tissue oxygenation spectrophotometric measurements. Another advantage is that different discrete wavelengths of light from the laser diodes 48 may be combined and interfaced to a single core multimode output optical fiber 44. Further, the different discrete wavelengths of light may pass through the optical fiber 44 in a homogeneous manner, such that the output light intensity from the single core multimode optical fiber 44 for all wavelengths is proportional to the input light intensity for all wavelengths, even if the input radiation profile or input NA are different for each wavelength. Still further, a homogeneous and relatively stable light output radiation profile or output NA may be achieved, even if the input radiation profile or input NA are lower and individually different for each wavelength. This is done by providing for relatively constant and high optical fiber modal filling by spreading the lower input modes to also fill higher modes until the optical fiber modes are filled; that is, a relatively large number or all of the modes or possible light guide pathways in the optical fiber 44 are utilized. Another advantage is that relatively homogeneous and stable light output intensity may be provided during rapid, transient, or gradual temperature changes, or during rapid, transient, or gradual optical fiber mechanical stress, such as fiber bending or vibration. A further advantage is that the optical sensors of a particular configuration used for biological tissue oxygenation measurement may be interchangeably utilized without having to be individually calibrated.

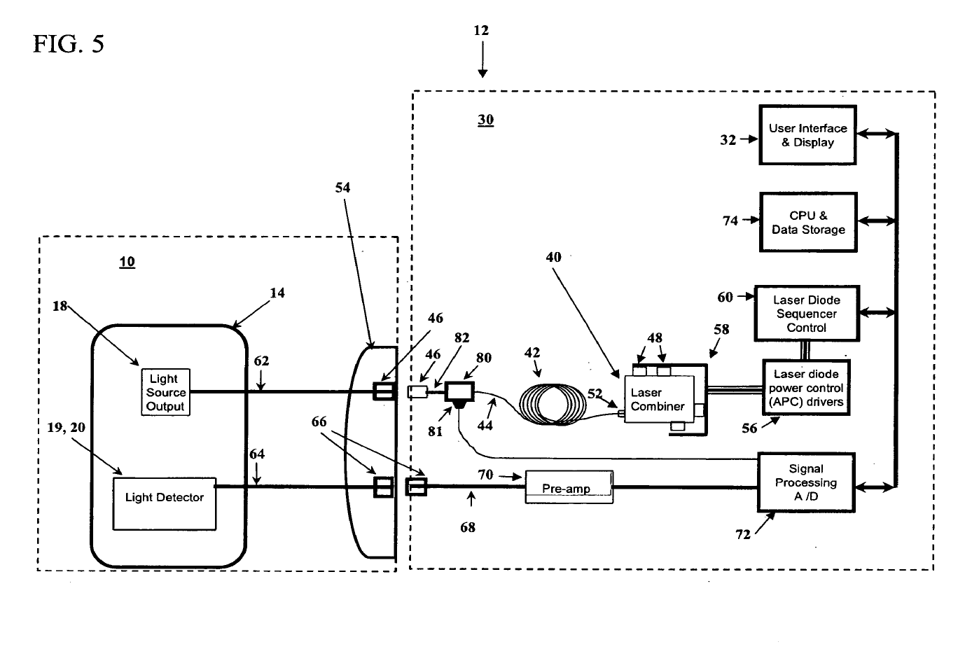

In an alternative embodiment, as shown in FIG. 5, an inline laser output intensity monitor 80 is placed after the optical fiber light stabilizer 42 to sample light using monitor photodiode 81, which functions in a manner similar to the laser output monitor photodiode 50 shown in FIG. 3. The inline laser output intensity monitor 80 contains a beam splitter that diverts a small percentage of light to monitor photodiode 81. The diverted light can be used as an input intensity (Io) signal within spectrophotometric type algorithms such as that described in U.S. Pat. No. 6,456,862. In addition, the inline laser output intensity monitor can be used to increase the output NA of the light emitting from the sensor by a combination of lenses or other optical means. In such cases, the optical fibers 82 and 62 of the sensor would have a higher NA than the optical fiber used for the optical fiber light stabilizer 42. A higher NA output of the sensor advantageously improves safety margin when using laser light for spectroscopic examination of biological tissue by decreasing intensity over a given surface area.

Although the present invention has been illustrated and described with respect to several preferred embodiments thereof, various changes, omissions and additions to the form and detail thereof, may be made therein, without departing from the spirit and scope of the invention. For example, the present spectrophotometric system and method has been described above in detail in terms of a cerebral oximeter useful to determine the oxygenation of biological tissue. The present spectrophotometric system and method is not limited to the described cerebral oximeter embodiment, however, and can be used alternatively to determine other tissue characteristics, or used to determine the presence of other substances that can be spectrophotometrically identified.

What is claimed is:

1. A spectroscopic system that monitors oxygenation levels in biological tissue, comprising:
a light source having a light output;
a predetermined length of optical fiber coupled to the light output of the light source, where the light output of the light source propagates through the optical fiber;
an optical fiber light stabilizer provided within the optical fiber, where the light output of the light source propagates through the optical fiber light stabilizer, and where the light stabilizer redistributes modes of the light output from the light source until an equilibrium mode distribution is established in the light output propagating within the optical fiber; and
a sensor coupled at an output of the light stabilizer, where the sensor provides the light output with the equilibrium mode distribution at an output of the spectroscopic system.

2. The spectroscopic system of claim 1, where the light source comprises a laser diode.

3. The spectroscopic system of claim 1, where the light source provides the light output at one or more predetermined wavelengths.

4. The spectroscopic system of claim 1, where the light source provides the light output at one or more predetermined wavelengths of light.

5. The spectroscopic system of claim 1, where the light source comprises a plurality of laser diodes.

6. The spectroscopic system of claim 1, where the light source comprises a plurality of laser diodes, each of the laser diodes providing a laser beam output at a predetermined wavelength, and where the spectroscopic system further comprises a laser beam combiner that combines together the laser beam outputs and provides the combined laser beam output to the optical fiber.

7. The spectroscopic system of claim 1, where the light stabilizer comprises a spool and a predetermined length of the optical fiber wrapped around the spool.

8. The spectroscopic system of claim 1, where the light stabilizer comprises a spool and a predetermined length of the optical fiber wrapped around the spool, where the spool has a radius that is at least approximately equal to a long term bend radius of the optical fiber wrapped around the spool.

9. The spectroscopic system of claim 1, further comprising a light detector that detects the light output from the light source.

10. The spectroscopic system of claim 1, further comprising a light detector that detects the light output from the light source and provides a detected light signal indicative thereof, and a signal processor that is responsive to the detected light signal to compensate for instability relating to the light output from the light source.

11. The spectroscopic system of claim 1, further comprising a light detector that detects the light output from the light source and provides a reference input intensity used for quantitative measurement methods in spectrophotometric applications.

12. The spectroscopic system of claim 11, whereas said light detector is a component of the light source.

13. The spectroscopic system of claim 11, whereas said light detector is used to monitor light output of the light stabilizer.

14. The spectroscopic system of claim 1 wherein the light output has a numeral aperture which is increased after reaching modal equilibrium within the light stabilizer by use of one or more lenses or other optical light bending means.

15. The spectroscopic system of claim 1, further comprising a control for providing power to the light source.

16. The spectroscopic system of claim 1, where the light source comprises a plurality of laser diodes, and where the spectroscopic system further comprises a control for providing power to the plurality of laser diodes in a predetermined sequence.

17. A spectrophotometric system that determines oxygenation levels in biological tissue and provides an indication of the determined oxygenation levels, comprising:
a plurality of laser diodes each providing a laser output signal at a predetermined wavelengths of light;
a device that combines the plurality of laser output signals into a combined laser output signal;
a predetermined length of optical fiber coupled to the combined laser output signal, where the combined laser output signal propagates through the optical fiber;
an optical fiber light stabilizer connected with the optical fiber, where the combined laser output signal propagates through the light stabilizer, and where the light stabilizer redistributes modes of the combined laser output signal until an equilibrium mode distribution is established in the combined laser output signal propagating within the optical fiber; and
a sensor coupled to the light stabilizer, where the sensor provides the combined laser output signal with the equilibrium mode distribution at an output of the spectrophotometric system.

18. The spectrophotometric system of claim 17, further comprising a light detector that detects the combined laser output signal and provides a detected light signal indicative thereof, and a signal processor that is responsive to the detected light signal to compensate for any instability relating to the combined laser output signal.

19. The spectroscopic system of claim 17, further comprising a light detector that detects the light output from the light source and provides a reference input intensity used for quantitative measurement methods in spectrophotometric applications.

20. The spectroscopic system of claim 19, whereas said light detector is a component of the light source.

21. The spectroscopic system of claim 19, whereas said light detector is used to monitor light output of the light stabilizer.

22. The spectroscopic system of claim 17 wherein the light output has a numeral aperture which is increased after reaching modal equilibrium within the light stabilizer by use of one or more lenses or other optical light bending means.

23. The spectrophotometric system of claim 17, where the light stabilizer comprises a spool and a predetermined length of the optical fiber wrapped around the spool

24. The spectrophotometric system of claim 17, where the light stabilizer comprises a spool and a predetermined length of the optical fiber wrapped around the spool, where the spool has a radius that is at least approximately equal to a long term bend radius of the optical fiber wrapped around the spool.

25. A spectrophotometric system that monitors biological tissue, comprising:
at least one laser diode that provides a laser output signal at a predetermined wavelength;
a predetermined length of optical fiber coupled to the laser output signal, where the laser output signal propagates through the optical fiber;
an optical fiber light stabilizer connected with the optical fiber, where the laser output signal propagates through the light stabilizer, and where the light stabilizer redistributes modes of the laser output signal until an equilibrium mode distribution is established in the laser output signal propagating within the optical fiber; and
a sensor coupled to the light stabilizer, where the sensor provides the laser output signal with the equilibrium mode distribution at an output of the spectrophotometric system.

26. The spectrophotometric system of claim 25, further comprising a plurality of laser diodes each providing a laser output signal at predetermined wavelengths of light, and a device that combines the plurality of laser output signals into a combined laser output signal, the predetermined length of optical fiber being coupled to the combined laser output signal.

27. The spectrophotometric system of claim 25, where the light stabilizer comprises a spool and a predetermined length of the optical fiber wrapped around the spool

28. The spectroscopic system of claim 25, further comprising a light detector that detects the light output from the light source and provides a reference input intensity used for quantitative measurement methods in spectrophotometric applications.

29. The spectroscopic system of claim 28, whereas said light detector is a component of the light source.

30. The spectroscopic system of claim 28, whereas said light detector is used to monitor light output of the light stabilizer.

31. The spectroscopic system of claim 25 wherein the light output has a numeral aperture which is increased after reaching modal equilibrium within the light stabilizer by use of one or more lenses or other optical light bending means.

32. The spectrophotometric system of claim 25, where the light stabilizer comprises a spool and a predetermined length of the optical fiber wrapped around the spool, where the spool has a radius that is at least approximately equal to the long term bend radius of the optical fiber wrapped around the spool.

33. The spectrophotometric system of claim 25, where the laser output signal at the output of the spectrophotometric system is provided to a human subject under test to monitor the oxygenation level in certain biological tissue of the human under test.

34. A method for spectrophotometrically determining oxygenation levels in biological tissue of a human subject under test, comprising the steps of:
providing a plurality of laser output signals each at a predetermined wavelength of light;
combining the plurality of laser output signals into a combined laser output signal;
providing the combined laser output signal to an optical fiber, where the combined laser output signal propagates through the optical fiber;
stabilizing the combined laser output signal within the optical fiber by redistributing modes of the combined laser output signal until an equilibrium mode distribution is established in the combined laser output signal propagating within the optical fiber;
emitting the combined laser output signal with established equilibrium mode distribution into the biological tissue of the human subject under test;
sensing the combined laser output signal after it has passed through the biological tissue of the human subject under test; and
determining the oxygenation levels in the biological tissue of the human subject under test from the sensed combined laser output signal.

35. A device for spectrophotometrically examining a subject's tissue, comprising:
a transmitter operable to transmit at least one light signal along at least three independent wavelengths;
at least one light signal detector operable to detect the light signal after passage through tissue being examined, and produce at least one detected signal corresponding to the light signal;
a secondary light signal detector to monitor the light signal prior to passage through the subject's tissue; and
a processor having an algorithm for examining the subject's tissue, which algorithm is adapted to process the detected signal along at least three wavelengths.

36. The device of claim 35, wherein the secondary light detector is operable to provide an output to the algorithm regarding the light signal transmitted by the transmitter.

37. The device of claim 36, wherein the secondary signal detector is operably disposed to receive at least a portion of the light signal.

38. The device of claim 37, wherein the secondary light detector includes a beam splitter.
